SEQUENCE LISTING (4 sequences)

SEQ ID NO: 1 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - DNA, 1102 bp, Homo sapiens
gcctggacagtcagcaaggaattgtctcccagtgcattttgccctcctggctgccaactctggctgctaaagcggctgccacctgctgcagtctacacagcttcgggaagaggaaaggaacctcagaccttccagatcgcttcctctcgcaacaaactatttgtcgcaggaataaagatggctgctgaaccagtagaagacaattgcatcaactttgtggcaatgaaatttattgacaatacgctttactttatagctgaagatgatgaaaacctggaatcagattactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagactagctattaaaatttcatgccgggcgcagtggctcacgcctgtaatcccagccctttgggaggctgaggcgggcagatcaccagaggtcaggtgttcaagaccagcctgaccaacatggtgaaacctcatctctactaaaaatactaaaaattagctgagtgtagtgacgcatgccctcaatcccagctactcaagaggctgaggcaggagaatcacttgcactccggaggtagaggttgtggtgagccgagattgcaccattgcgctctagcctgggcaacaacagcaaaactccatctcaaaaaataaaataaataaataaacaaataaaaaattcataatgtg

SEQ ID NO: 3 - DNA, 31 bp, Artificial Sequence
  primer
catcgcgagcccaaactttggccgacttcac

SEQ ID NO: 4 - DNA, 48 bp, Artificial Sequence
  primer
gttagatctctaatggtgatgatggtgatgactttgatgtaagttagt

(19) United States
(12) Patent Application Publication
(10) Pub. No.: US 2001/0044418 A1
(43) Pub. Date: Nov. 22, 2001

(54) Treatment of allergies

(76) Inventors: Shoshana Levy, Stanford, CA (US); Rosemarie H. Dekruyff, Stanford, CA (US); Dale Teiji Umetsu, Stanford, CA (US); Holden Maecker, Mountain View, CA (US)

Correspondence Address: PAMELA J. SHERWOOD, BOZICEVIC, FIELD & FRANCIS LLP, 200 Middlefield Road, Suite 200, Menlo Park, CA, 94025, US

(21) Appl. No.: 09/798,075

(22) Filed: Mar. 2, 2001

Related U.S. Application Data
(60) Provisional application No. 60/188,311, filed on Mar. 10, 2000.

(51) Int. Cl. A61K 48/00; A61K 38/20

(52) U.S. Cl. 514/44; 424/85.2

(57) ABSTRACT

Allergic and other immune disorders associated with antigen specific T cells are treated by administering a vaccine comprising sequences of a fusion protein of IL-18 and antigen. The methods are particularly useful in decreasing an established antigen specific IgE immune response. Conditions of particular interest include asthma, allergic rhinitis, IgE-mediated anaphylactic reactions to insect stings, and other allergic conditions.

### GOVERNMENT SUPPORT

This work was supported at least in part by a grant from the National Institutes of Health. The Government may have certain rights in this invention.

### INTRODUCTION

### BACKGROUND

The prevalence of allergic asthma has dramatically increased over the last two decades and is a major public health concern. Asthma is characterized by airway hyperresponsiveness (AHR) to a variety of specific and nonspecific stimuli, chronic pulmonary inflammation with eosinophilia, excessive mucus production and high serum IgE levels. The pathology in asthma results from excessive production of IL-4, IL-5 and IL-13 by CD4⁺ Th2 cells (Wills-Karp et al. (1998) Science 282:2258; Umetsu and DeKruyff (1997) J. Aller. Clin. Immunol. 100:1).

Current treatments for asthma are not satisfactory and disease prevention is not possible. Therapies such as inhaled corticosteroids, anti-leukotrienes or β2-agonists, focus rather on symptom relief, reduction or neutralization of effector molecules and inflammatory mediators. This approach, while effective for acute disease and for relieving symptoms, however, has limited long term salutary effects, since the environmental factors that cause and precipitate asthma are not eliminated, and patients redevelop symptoms of asthma when these medications are discontinued.

One approach to allergic diseases is immunotherapy. Immunotherapy has proven to be effective when used properly, and it is hoped that advances in immunologic intervention will further improve the efficacy. Modification of allergens, and the use of cytokines, may succeed in shutting down production of specific IgE and thus cure symptomatic allergies. Alternative approaches have attempted to use cytokines to shift the immune response. IL-12, a heterodimeric cytokine produced by macrophages and dendritic cells, is potent in driving the development of Th1 cytokine synthesis in naive and memory CD4+ T cells. However, several in vivo studies have demonstrated that rIL-12 as an adjuvant, while enhancing IFN-_{γ} synthesis, in some cases paradoxically also increases IL-4 and IL-10 synthesis in antigen primed CD4⁺ T cells.

Immunotherapy may also utilize DNA-based immunization. Vaccination with allergen in the form of naked plasmid cDNA stimulates allergen-specific immune responses with a Th1 bias, and amplifies the expansion of CD4⁺ T cells producing IFN_{γ} and of cytotoxic CD8⁺ T cells (Roman et al. (1997) Springer Semin Immunopathol 19:223). The key feature of this strategy is that injection of plasmid DNA encoding a specific antigen produces an allergen-specific protective immune response, that should be reinforced by natural exposure to the allergen, thus conferring long-lasting protection (Donnelly et al. (1997) Annu Rev Immunol 15:617).

Immunotherapy is feasible only if therapies are developed that reverse ongoing airway hyperreactivity and reverse the ongoing allergic inflammatory process, which plays a critical role in the pathogenesis of asthma (Martinez et al. (1995) New Engl. J. Med. 332:133-8). Previous studies with DNA immunization strategies demonstrated its success in preventing the development of antigen-specific IgE synthesis and airway hyperresponsiveness (Hsu et al. (1996) Nat. Med. 2:540. However, successful reversal of ongoing AHR with DNA vaccination has not been reported. Thus, improvement of gene vaccination methodologies is required for successful clinical application of DNA vaccination to symptomatic patients with allergic asthma.

The immune response to an antigen is affected by the presence or absence of cytokines. Interleukin (IL)-18 is a cytokine with profound effects on T-cell activation. IL-18 plays an important role in the T-cell-helper type 1 (Th1) response, primarily by its ability to induce IFN-_{γ} production in T cells and natural killer (NK) cells. IL-18 induces gene expression and synthesis of tumor necrosis factor (TNFα), IL-1β, Fas ligand, and several chemokines.

IL-18 and IL-1β share primary amino acid sequences of the so-called “signature sequence” motif and are similarly folded as all beta-pleated sheet molecules. Also similar to IL-1β, IL-18 is synthesized as a biologically inactive precursor molecule lacking a signal peptide which requires cleavage into an active, mature molecule by the intracellular cysteine protease called IL-1β-converting enzyme (ICE, also called caspase-1).

IL-18 binds a receptor complex comprising a binding chain termed IL-18Rα, a member of the IL-1 receptor family previously identified as the IL-1 receptor-related protein (IL-1Rrp), and a signaling chain, also a member of the IL-1R family. The receptor complex recruits the IL-1R-activating kinase (IRAK) and TNFR-associated factor-6 (TRAF-6) which phosphorylates nuclear factor ₖB (NFₖB)-inducing kinase (NIK) with subsequent activation of NFₖB.

In contrast with drug therapy, immunotherapy could result in long-term, favorable alteration of the patient's immunologic status. Immunological changes that have been described after immunotherapy include an initial rise in specific serum IgE, followed by a fall, and a rise in specific IgG (“blocking antibody”). Immunotherapy leads to a reduction in mediator release from mast cells in vitro, alterations in lymphocyte subsets, and a downregulation of IL-4 production from T cells (Secrist et al. (1993) J. Exp. Med. 178: 2123-2130). Several studies have shown a reduction in inflammation and a decrease in bronchial hyper-responsiveness after immunotherapy.

Current therapy for asthma aims to suppress inflammation but does not address the initiating event in allergic asthma. By altering the immune response to allergen, it may be possible to control the trigger of asthma, and of other allergic disorders.

Relevant Literature

Ushio et al. (1996) J Immunol 156(11):4274-9, describe the cloning of the cDNA for human IFN-gamma-inducing factor (IL-18). Okamura et al. (1995) Nature 378:88-91 describe the identification of mouse IL-18. The combined activity of IL-12 and IL-18 on IgE synthesis is disclosed by Yoshimoto et al. (1997) Proc. Natl. Acad. Sci. USA. 94:3948-3953. Co-delivery of IL-18 gene adjuvant is described by Kim et aL (1999) J. Med. Primatol. 28:214-223. Kumano et al. (1999) Am. J. Respir. Crit. Care Med. 160(3):873-878 suggest that IL-18 exacerbates asthma through the recruitment of eosinophils.

Conjugates of an antigen and cytokine are described by Levy et al. International patent application WO94/08601. Fusion proteins of GM-CSF and antigen are described by Price, International patent application WO96/01903. The use of an IL-12 fusion protein is discussed in Kim et al. (1997) J. Immunol. 158:4137. Other cytokine fusion constructs are disclosed in Maecker et al. (1997) Vaccine 15:1687.

Weber (1997) JAMA 278(22):1881-1887 reviews immunotherapy with allergens. Bousquet et al. (1991) J. Aller. Clin. Immunol. 99:43-53 provide evidence for immunotherapy efficacy. Soderlund et al. (1997) Immunol Lett 57(1-3):177-181 discuss allergen induced cytokine profiles in type I allergic individuals before and after immunotherapy. Nelson (1997) Allergy Asthma Proc 18(3):157-162; and Creticos et al. (1996) N Engl J Med 334(8):501-506, review the efficacy of immunotherapy for asthma exacerbated by seasonal ragweed exposure. Gavett et al. (1995) J. Exp. Med. 182:1527-1536 disclose a role for IL-12 in asthma immunotherapy.

### SUMMARY OF THE INVENTION

Methods are provided for the treatment of allergic and other immune disorders associated with antigen specific T cells. The subject methods are useful in decreasing an established antigen specific IgE immune response. Conditions of particular interest include asthma, allergic rhinitis, IgE-mediated anaphylactic reactions to insect stings, and other allergic conditions. The methods of the invention utilize a vaccination protocol to introduce a fusion peptide of interleukin 18 (IL-18) and an antigen. In a preferred embodiment the antigen is an allergen. The fusion peptide may be introduced a polypeptide or as a DNA construct encoding the peptide. The IL-18 fusion construct provides a unique capacity to effectively reverse established airway hyperresponsiveness in asthma patients.

### BRIEF DESCRIPTION OF THE DRAWINGS

FIG. 1A is a diagram of an immunization vector expressing an OVA-IL-18 fusion construct. FIG. 1B shows the bioactivity of the in vitro secreted OVA-IL-18 fusion protein. FIG. 1C depicts an immunization scheme for prevention of AHR by DNA vaccination (top) or reversal of established AHR by DNA vaccination (bottom).

FIG. 2A is a graph depicting the prevention of induction of AHR by DNA vaccination. FIG. 2B is a graph depicting the level of IFN_{γ} levels in the supernatants from spleen cells of immunized animals. FIG. 2C is a graph depicting the OVA-specific IgE production from sera of the mice collected within 5 days of the last challenge.

FIG. 3 shows the antigen specificity of IFN_{γ} production in response to OVA-IL-18 DNA vaccination.

FIG. 4 depicts the inhibition of AHR development by OVA-IL-18 DNA vaccination depends upon CD8⁺ cells and IFN_{γ}.

FIGS. 5A and 5B show the reversal of AHR by OVA-IL-18 DNA vaccination. Only OVA-IL-18 DNA, and not OVA DNA alone, protected against AHR under these conditions. Mice were sensitized to OVA and then immunized twice with either OVA DNA or OVA-IL-18 DNA or (5B) OVA DNA, IL-18 DNA, OVA-IL-18 DNA or a combination of OVA DNA+IL-18 DNA. AHR in response to methacholine challenge was measured as above. Only OVA-IL-18 DNA, and not OVA DNA alone, reversed established AHR under these conditions. FIG. 5C shows the recruitment of specific cell types into the airways of immunized mice. FIG. 5D depicts the levels of IFN_{γ} and IL-4 secretion by bronchial LN cells from the vaccinated mice.

### DESCRIPTION OF THE SPECIFIC EMBODIMENTS

The subject methods provide a means for upregulating a Th1 immune response, e.g. T cell mediated response to tumor antigens, pathogens, etc. The methods are particularly beneficial in the therapeutic treatment and investigation of allergic responses. In response to this treatment, the effects of the allergic response are decreased, which effects may include synthesis of specific cytokines, including _{γ}-IFN; and physiological effects such as bronchial hyperreactivity, anaphylaxis, etc. The synthesis of allergen specific IgE antibodies is decreased, thereby alleviating the symptoms of diseases such as asthma, allergic rhinitis, IgE-mediated anaphylactic reactions to insect stings, and other allergic conditions.

Immunotherapy is performed by administering a fusion polypeptide comprising an antigen of interest, and IL-18. Preferably the method uses DNA vaccination as the route of administration, although administration of the fusion polypeptide may also find use. The vaccine reduces antigen-specific IgE production and increases synthesis of IFN-_{γ}.

Of particular interest is the treatment of asthma. Asthma is a respiratory disorder characterized by airway hyperreactivity and inflammation, and is associated with high serum IgE and overproduction of interleukin (IL)-4, IL-5 and IL-13 by allergen-specific Th2 cells. Two doses of the vaccine have been shown to significantly reduce airway hyperreactivity and reverse established airway hyperreactivity when given late after allergen-sensitization. The immunotherapy mediates immune deviation from pathological response towards a protective immune response in peripheral lymphoid tissues and in the lungs, and is effective in the treatment of patients with established asthma and allergic disease.

The subject methods of suppressive immunization are used for prophylactic or therapeutic purposes. As used herein, the term “treating” is used to refer to both prevention of disease, and treatment of pre-existing conditions. The treatment of ongoing disease, where the suppressive immunotherapy stabilizes or improves the clinical symptoms of the patient, is of particular interest.

Definitions

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

Interleukin-18 (IL-18) fusion: The fusion construct of the invention comprises the following components: (a) an IL-18 subunit; (b) an antigen subunit; and (c) a linker, which may be a peptide bond directly between the two subunits, or may be a polypeptide linker. Preferably a mature form of the IL-18 protein is encoded, although the complete cDNA sequence, including the propeptide, may be used for some purposes. The antigen and IL-18 subunits may be in either order. The fusion proteins of the invention have IL-18 activity, herein defined as being at least one biological property of naturally occurring IL-18. Such properties include the ability to stimulate interferon-_{γ} expression, and the decrease of IgE synthesis.

For use in the present invention, the nucleic acid encoding the fusion polypeptide may be used in a DNA vaccination protocol, or the fusion polypeptide may be administered as a protein vaccine.

The cDNA sequence of human IL-18 is provided for convenience in the sequence listing, and may be accessed in public databases, e.g. Genbank accession number D49950. This sequence is the preferred coding sequence of the invention. However, the invention is not limited to the use of this sequence in constructs of the invention. Also of use are closely related variant sequences that have the same biological activity, or substantially similar biological activity.

Variant sequences encode protein subunits which, when present in a fusion protein of the invention, give the fusion protein one or more of the biological properties of IL-18 as described above. DNA sequences of the invention may differ from a native IL-18 sequence by the deletion, insertion or substitution of one or more nucleotides, provided that they encode a protein with the appropriate biological activity as described above. Similarly, they may be truncated or extended by one or more nucleotides. Alternatively, DNA sequences suitable for the practice of the invention may be degenerate sequences that encode the naturally occurring IL-18 protein. Typically, DNA sequences of the invention have at least 70%, at least 80%, at least 90%, at least 95% or at least 99% sequence identity to a native IL-18 coding sequence. They may originate from any species, though DNAs encoding human proteins are preferred. Variant sequences may be prepared by any suitable means, as known in the art.

With respect of substitutions, conservative substitutions are preferred. Typically, conservative substitutions are substitutions in which the substituted amino acid is of a similar nature to the one present in the naturally occurring protein, for example in terms of charge and/or size and/or polarity and/or hydrophobicity. Similarly, conservative substitutions typically have little or no effect on the activity of the protein. Proteins of the invention that differ in sequence from naturally occurring IL-18 may be engineered to differ in activity from naturally occurring IL-18. Such manipulations will typically be carried out at the nucleic acid level using recombinant techniques known in the art.

Within the fusion protein coding region, a linker sequence may join the antigen coding sequence and the IL-18 coding sequence, using any suitable sequence, as long as the fusion protein has IL-18 activity, as defined above. Specifically, the linker sequence may encode a sequence of suitable length to allow both subunits to fold correctly, as they do in nature or substantially as they do in nature, in order to retain the biological or antigenic activity of the subunits. Preferably, an encoded linker comprises amino acids that do not have bulky side groups and therefore do not obstruct the folding of the protein subunits. Further, it is preferred to use uncharged amino acids in the linker. A linker may be any suitable length, preferably, the linker is not more than about 10 amino acids in length, and may comprise only a peptide bond between the two subunits.

The antigen subunit may include viral, prokaryotic and eukaryotic antigens, including but not limited to antigens derived from bacteria, fungi, protozoans, parasites and tumor cells. Potential tumor antigens for immunotherapy include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and over-expressed self antigens, e.g. her-2/neu; carcinoembryonic antigen, muc-1, and the like.

Of particular interest are antigens that elicit an allergic response. These include, for example, proteins found in food, such as strawberries, peanuts, milk proteins, egg whites, etc. Other allergens of interest include various airborne antigens, such as grass pollens, animal danders, house mite feces, etc. Molecularly cloned allergens include Dermatophagoides pteryonyssinus (Der P1); Lol pl-V from rye grass pollen; a number of insect venoms, including venom from jumper ant Myrmecia pilosula; Apis millifera bee venum phospholipase A2 (PLA.sub.2) and antigen 5S; phospholipases from the yellow jacket Vespula maculifrons and the white faced hornet Dolichovespula maculata; a large number of pollen proteins, including birch pollen, ragweed pollen, Paro1 (the major allergen of Parietaria officinalis) and the cross-reactive allergen Parjl (from Parietaria judaica), and other atmospheric pollens including Olea europaea, Artemisia sp., gramineae, etc. Other allergens of interest are those responsible for allergic dermatitis caused by blood sucking arthropods, e.g. Diptera, including mosquitos (Anopheles sp., Aedes sp., Cuffseta sp., Culex sp.); flies (Phlebotomus sp., Culicoides sp.) particularly black flies, deer flies and biting midges; ticks (Dermacenter sp., Omithodoros sp., Otobius sp.); fleas, e.g. the order Siphonaptera, including the genera Xenopsylla, Pulex and Ctenocephalides.

Reviews of molecularly cloned allergens include Chapman et al. (1997) Allergy 52(4):374-9 “Recombinant mite allergens”; King (1996) Toxicon. 34(11-12):1455-88, “Immunochemical studies of stinging insect venom allergens”; Becker et al. (1995) Int Arch Allergy Immunol. 107(1-3):242-4, “Molecular characterization of timothy grass pollen group V allergens”; and Scheiner et aL. (1994) Arb Paul Ehrlich Inst Bundesamt Sera Impfstoffe Frankf A M. (87):221-32, “Molecular and functional characterization of allergens: basic and practical aspects”.

The vaccine may be formulated with one or a cocktail of IL-18-antigen fusion sequences. While it has been found that a single sequence is capable of suppressing a response to multiple epitopes, it may be desirable in some cases to include multiple sequences, where each encodes a different epitope. For example, see Leadbetter et al. (1998) J. Immunol. 161:504-512. A formulation comprised of multiple sequences of distinct epitopes may be used to induce a more potent and/or sustained suppressive response.

Nucleic acid constructs: Nucleic acid constructs of the invention comprise a coding sequence for the fusion polypeptide; and a promoter operably linked to the coding region. Any suitable promoter may be used to control the expression of the nucleic acid of the invention, preferably a strong promoter. In general, it is preferred to use a viral promoter or a promoter adapted to function in a cell into which the constructs are to be introduced. Thus, in the case of a human cell, for example, it is preferred to use viral promoters, especially promoters derived from viruses that infect humans, or promoters derived from human genes. Tissue-specific promoters may also be used, e.g. muscle specific promoters. Optionally, a promoter is used in combination with any suitable enhancer. Preferred promoters include the cytomegalovirus (CMV) promoter, optionally in combination with the CMV enhancer; the human β-actin promoter; the simian virus 40 (SV40) early gene promoter; the Rous sarcoma virus (RSV) promoter; and the retroviral long terminal repeat (LTR) promoter.

The construct may comprise a polyadenylation signal and/or a transcriptional terminator downstream of the fusion protein. Any suitable transcriptional terminator known in the art may be used. The construct may also comprise one or more selectable marker genes, e.g. antibiotic resistance genes, to allow selection of cells transformed or transfected cells with the construct.

Plasmid vectors are preferred, particularly where the vector includes immunostimulatory DNA sequence, as discussed below. Alternatively, viral vectors may be used, including adenoviruses, adeno-associated viruses (AAVs), retroviruses, pseudotyped retroviruses, herpesviruses, vaccinia viruses, etc. Viral vectors of the invention are preferably disabled, e.g. replication-deficient.

Immunostimulatory DNA sequences: In addition to the specific epitopes and polypeptides of autoantigens, the immune response may be enhanced by the inclusion of CpG sequences, as described by Krieg et al. (1998) Trends Microbiol. 6:23-27, and helper sequence, King et al. (1998) Nat. Med. 4:1281-1286. Biological effects of DNA motifs like unmethylated CpG dinucleotides in particular base contexts (CPG-S motifs) may modulate innate immune responses when injected to animals.

An “immunostimulatory oligonucleotide” refers to an oligonucleotide that contains a cytosine/guanine dinucleotide sequence and stimulates maturation and activation of DC. An immunostimulatory oligonucleotide of interest may be between 2 to 100 base pairs in size and typically contain a consensus mitogenic CpG motif represented by the formula: 5′ X₁ X₂ CGX₃ X₄ 3′, where C and G are unmethylated, X₁, X₂, X₃ and X₄ are nucleotides and a GCG trinucleotide sequence is not present at or near the 5′ and 3′ termini (see U.S. Pat. No. 6,008,200, Krieg et a., issued Dec. 28, 1999, herein incorporated by reference).

Preferably the immunostimulatory sequences range between 8 to 40 base pairs in size. The dose and protocol for delivery will vary with the specific agent that is selected. Typically the immunostimulatory sequences are included in the backbone of the vector that encodes the IL-18 antigen fusion polypeptide.

Allergy, or Atopy is an increased tendency to IgE-based sensitivity resulting in production of specific IgE antibody to an immunogen, particularly to common environmental allergens such as insect venom, house dust mite, pollens, molds or animal danders. Allergic responses are antigen specific. The immune response to the antigen is further characterized by the over-production of Th2-type cytokines, e.g. IL-4, IL-5 and IL-10, by the responding T cells. The sensitization occurs in genetically predisposed people after exposure to low concentrations of allergen; cigarette smoke and viral infections may assist in the sensitization process.

Included in the group of patients suffering from atopy are those with asthma associated allergies. About 40% of the population is atopic, and about half of this group develop clinical disease ranging from trivial rhinitis to life-threatening asthma. After sensitization, continuing exposure to allergens leads to a significant increase in the prevalence of asthma. Ninety per cent of children and 80% of adults with asthma are atopic. Once sensitization has occurred, re-exposure to allergen is a risk factor for exacerbations of asthma. Effective management of allergic asthma includes pharmacological therapy and allergen avoidance. The specific physiological effects of asthma associated allergies include airway inflammation, eosinophilia and mucus production, and antigen-specific IgE and IL-4 production.

In addition to allergies affecting human populations, non-human mammals are also known to suffer from allergic conditions. Fleas, Ctenocephalides felis felis and others, are now recognized as a major cause of physiological disorders among mammals. These insects are ectoparasites that attack dogs, cats, and humans. Certain species (i.e., dogs and cats), and individuals of these species are more allergic to fleabites than others, resulting in a clinical disorder called flea allergy dermatitis (FAD) or flea bite hypersensitivity.

A test allergen may be used to determine whether an individual is hypersensitive to a particular compound, and may be any antigen suspected of causing a hypersensitive immune response. The selection of allergens for immunotherapy will be based on standard tests performed on the patient. A review of allergen skin tests in current use are reviewed by Gordon (1998) Otolaryngol Clin North Am 31(1):35-53. All current skin tests are capable of detecting allergic hypersensitivity, but the tests differ in their sensitivity, specificity, safety, reproducibility, and applications.

Other conventional tests for hypersensitivity include a skin test, where the allergen is injected intradermally. Contact with the allergen results in mast cell degranulation and release of histamines, heparin, eosinophil and neutrophil chemotactic factors, leukotrienes and thromboxanes, etc. A hypersensitive response will cause rapid production of a wheal and erythema within 30 minutes.

Allergen immunotherapy, or hyposensitization is the administration of allergenic extracts as antigens at periodic intervals, usually on an increasing dosage scale to a dosage that is maintained as maintenance therapy. Indications for immunotherapy are determined by appropriate diagnostic procedures coordinated with clinical judgement and knowledge of the patient history of allergic disease. Allergen immunotherapy is performed by providing injections of the allergen to the allergic subject on a regular basis, with the goal of reducing the symptoms and signs of an allergic reaction or prevention of future anaphylaxis against antigens such as insect venom, penicillin, etc. This is usually done initially with low doses, with gradual dosage increases over a period of weeks.

Immunotherapy is specific to the allergen injected. It results in the following immunologic changes: a shift in T cell response from a Th1-type response to a Th2-type response with corresponding changes in cytokine production, decreased allergen-specific IgE, increased allergen-specific IgG, decreased inflammatory cells, decreased mediators of inflammation and decreased histamine-releasing factors. These changes result in decreased reactivity to the allergen in the target organ.

Allergen immunotherapy is appropriate for the following indications: Severe, seasonal (lasting 2 or more years) or perennial, IgE-dependent allergic rhinoconjunctivitis in which optimal allergen avoidance and medication have not been sufficiently effective in controlling symptoms; IgE-mediated allergic asthma; particularly where there is a clear temporal association between exposure to the allergen and signs and symptoms of asthma, and those in which symptoms have occurred during two or more allergy seasons in successive years; IgE-mediated asthma caused by house dust mites or ragweed pollen may be treated with allergen immunotherapy; IgE-mediated anaphylactic reactions to insect stings. Immunotherapy with venom from yellow jackets, yellow hornets, white-faced hornets, wasps and honey-bees, and with whole-body extracts of fire-ants, is effective. Flea allergy dermatitis, particularly in pets such as cats and dogs may also be treated with the subject methods.

Asthma, as defined herein, is reversible airflow limitation in a patient over a period of time. Asthma is characterized by the presence of cells such as eosinophils, mast cells, basophils, and CD25+ T lymphocytes in the airway walls. There is close interaction between these cells, because of the activity of cytokines which have a variety of communication and biological effector properties. Chemokines attract cells to the site of inflammation and cytokines activate them, resulting in inflammation and damage to the mucosa. With chronicity of the process, secondary changes occur, such as thickening of basement membrane and fibrosis. The disease is characterized by increased airway responsiveness to a variety of stimuli, and airway inflammation. A patient diagnosed as asthmatic will generally have multiple indications over time, including wheezing, asthmatic attacks, and a positive response to methacholine challenge, i.e. a PC₂₀ on methacholine challenge of less than about 4 mg/ml. Guidelines for diagnosis may be found in the National Asthma Education Program Expert Panel. Guidelines for diagnosis and management of asthma. National Institutes of Health, 1991; Pub. #91-3042.

Methods of Immunotherapy

The present invention provides novel therapeutic compositions and methods of vaccination that enhance the hyposensitization (hyper? Check with Dr. Umetsu) procedures used in allergen immunotherapy. Such compositions elicit a an immune response that decreases adverse effects of allergic responses, and can reverse on-going airway hyperresponsiveness.

The vaccine may be formulated with one or a cocktail of antigen sequences, which may be on the same or different vectors. The DNA vectors are suspended in a physiologically acceptable buffer, generally an aqueous solution e.g. normal saline, water, etc. Stabilizing agents, wetting and emulsifying agents, salts for varying the osmotic pressure or buffers for securing an adequate pH value, and skin penetration enhancers can be used as auxiliary agents. The DNA will usually be present at a concentration of at least about 1 ng/ml and not more than about 10 mg/ml, usually at about from 100 μg to 1 mg/ml.

The vaccine may be fractionated into two or more doses, of at least about 1 μg, more usually at least about 100 μg, and preferably at least about 1 mg per dose, administered from about 4 days to one week apart. In some embodiments of the invention, the individual is subject to a series of vaccinations to produce a full, broad immune response. According to this method, at least two and preferably four injections are given over a period of time. The period of time between injections may include from 24 hours apart to two weeks or longer between injections, preferably one week apart. Alternatively, at least two and up to four separate injections are given simultaneously at different parts of the body.

The DNA vaccine is injected into muscle or other tissue subcutaneously, intradermally, intravenously, orally or directly into the spinal fluid. Of particular interest is injection into skeletal muscle. The genetic vaccine may be administered directly into the individual to be immunized or ex vivo into removed cells of the individual which are reimplanted after administration. By either route, the genetic material is introduced into cells which are present in the body of the individual. Alternatively, the genetic vaccine may be introduced by various means into cells that are removed from the individual. Such means include, for example, transfection, electroporation and microprojectile bombardment. After the genetic construct is taken up by the cells, they are reimplanted into the individual.

Otherwise non-immunogenic cells that have genetic constructs incorporated therein can betaken from one individual and implanted into another. Electroporation has now been used very effectively in vivo (Mir et. al PNAS 96: 4262, 1999; Aihara and Miyazaki Nature Biotechnology 16:867, 1998) and may be developed in the future for use in human.

An example of intramuscular injection may be found in Wolff et al. (1990) Science 247:1465-1468. Jet injection may also be used for intramuscular administration, as described by Furth et al. (1992) Anal Biochem 205:365-368. The DNA may be coated onto gold microparticles, and delivered intradermally by a particle bombardment device, or “gene gun”. Microparticle DNA vaccination has been described in the literature (see, for example, Tang et al. (1992) Nature 356:152-154). Gold microprojectiles are coated with the vaccine cassette, then bombarded into skin cells.

The genetic vaccines are formulated according to the mode of administration to be used. One having ordinary skill in the art can readily formulate a genetic vaccine that comprises a genetic construct. In cases where intramuscular injection is the chosen mode of administration, an isotonic formulation is used. Generally, additives for isotonicity can include sodium chloride, dextrose, mannitol, sorbitol and lactose. Isotonic solutions are preferred. Stabilizers include gelatin and albumin.

According to the present invention, prior to or contemporaneously with administration of the genetic construct, cells may be administered with cell stimulating or cell proliferative agents, which terms are used interchangeably and refer to compounds that stimulate cell division and facilitate DNA and RNA uptake.

Bupivacaine or compounds having a functional similarity may be administered prior to or contemporaneously with the vaccine. Bupivacaine is a homologue of mepivacaine and related to lidocaine. It renders muscle tissue voltage sensitive to sodium challenge and effects ion concentration within the cells. In addition to bupivacaine, mepivacaine, lidocaine and other similarly acting compounds, other contemplated cell stimulating agents include lectins, growth factors, cytokines and lymphokines such as platelet derived growth factor (PDGF), G-CSF, GM-CSF, epidermal growth factor (EGF) and IL-4. About 50 μl to about 2 ml of 0.5% bupivacaine-HCl and 0.1% methylparaben in an isotonic pharmaceutical carrier may be administered to the site where the vaccine is to be administered, preferably, 50 μl to about 1500 μl, more preferably about 1 ml. The genetic vaccine may also be combined with collagen as an emulsion and delivered intraperatonally. The collagen emulsion provides a means for sustained release of DNA. 50 μl to 2 ml of collagen are used.

The efficiency of DNA vaccination may be improved by injection of cardiotoxin into the tissue about one week prior to the vaccination, as described by Davis et al. (1993) FEBS Lett. 333:146-150, and in the examples. The cardiotoxin stimulates muscle degeneration and regeneration. The muscle is injected with from about 0.1 to 10 μM of cardiotoxin dissolved in a pharmacologically acceptable vehicle.

It should be emphasized that immunotherapy schedules are individualized and fixed schedules are not recommended. Allergy injections rarely go on “forever” but can usually be stopped after a patient has experienced no allergic symptoms and has required no medication for 18 -24 consecutive months while on the maintenance schedule. Duration of treatment for the average patient is 3 to 5 years but could be longer in certain clinical settings. If symptoms recur after a 6 to 12 months observation period following discontinuation of immunotherapy, re-evaluation is warranted.

Where the fusion protein is administered as a polypeptide, recombinant techniques may be employed to create a nucleic acid encoding the peptide of interest, and to express that peptide under desired conditions (e.g., in a host cell or an in vitro expression system from which it can readily be purified). The fusion protein may be formulated with Freund [symbol] s incomplete adjuvant, with QS21, or with others. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacial, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters or higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant is placed in proximity to the site of response, where applicable, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host. Unit dosage forms for injection or intravenous administration may comprise the compound of the present invention in a composition as a soluble in sterile water, normal saline or another pharmaceutically acceptable carrier.

Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 μg to 100 milligrams per kg weight of subject per administration. A typical dosage may be one sub-cutaneous injection administered at weekly or semi-weekly intervals. A time-release effect may be obtained by capsule materials that dissolve at different pH values, by capsules that release slowly by osmotic pressure, or by any other known means of controlled release.

The immunization protocol may be repeated for extended periods of time. Treatment will generally be continued until there is a substantial reduction in the allergic effects, e.g. at least about 50% decrease in the serum concentration of allergen specific IgE, and decreased bronchial hyperreactivity as measured by methacholine challenge, etc. Monitoring of IgE concentration will be performed in accordance with standard techniques, e.g. ELISA, RIA, etc. The cytokines produced by responding T cells may also be monitored, where therapy results in increased levels of _{γ}-IFN, and decreased levels of IL-10, IL-4 and IL-5.

Those of skill in the art will readily appreciate that dose levels can vary as a function of the specific allergen, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

Mammalian species susceptible to allergic conditions include canines and felines; equines; bovines; ovines; etc. and primates, particularly humans. Animal models, particularly small mammals, e.g. murine, lagomorpha, etc. may be used for experimental investigations. Animal models of interest include those involved with the production of antibodies having isotypes associated with Th1 responses, influenced by IL-18 production.

### Experimental

The following examples are put forth so as to provide those of ordinary skill in the art with a complete disclosure and description of how to make and use the subject invention, and are not intended to limit the scope of what is regarded as the invention. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade; and pressure is at or near atmospheric.

### Example 1

Methods

Animals. BALB/cByJ mice were obtained from Jackson Laboratory (Bar Harbor, Me.). Animals were used between 6 and 10 weeks of age and were age and sex matched within each experiment. The Stanford University Committee on Animal Welfare approved all animal protocols.

Monoclonal Antibodies and Reagents. Monoclonal antibodies were purified from ascites fluid by ammonium sulfate precipitation and ion-exchange chromatography. The following hybridomas were used: R46A2 (anti-IFN_{γ}), obtained from ATCC, Rockville, Md.; XMG1.2 (anti-IFN_{γ}); BVD4-1D11 (anti-IL-4) and BVD6-24G2 (anti-IL-4), DNAX Research Institute, Palo Alto, Calif.; 53.6.7 (anti-CD8); EM95 (rat anti-mouse IgE). Anti-OVA mAbs and biotinylated anti-OVA mAb were produced as described previously (Kim et aL (1997), supra.)

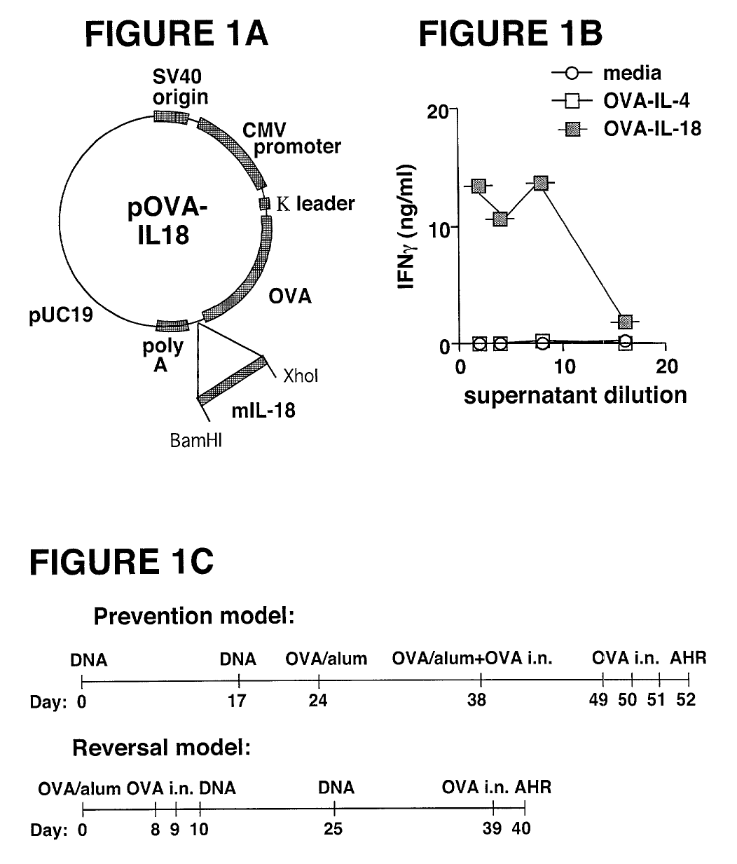

DNA Constructs. A series of plasmids expressing OVA fused to various cytokines was produced in our laboratory and has been described in Maecker et al., supra. One of these plasmids, expressing OVA-IL-4, was digested with XhoI and BamHI to excise the IL-4 portion of the insert. The remainder of the plasmid was ligated to a similarly digested PCR product encoding mature murine IL-18 (FIG. 1A). This sequence was isolated by PCR amplification of cDNA produced from RNA of C3H mouse splenocytes activated with ConA. The forward PCR primer, which incorporated an XhoI site, was: (SEQ ID NO:3) 5′ CATCGCGAGCCCAAACTTTGGCCGACTTCAC 3′. The reverse PCR primer, which incorporated a BamHI site, a stop codon, and a hexahistidine tag, was: 5′ (SEQ ID NO:4) GTTAGATCTCTMTGGTGATGATGGTGATGACTTTGATGTAA GTTAGT 3′. The ligated plasmid (FIG. 1A) was electroporated into E. coli and purified from a large-scale culture by alkaline lysis and CsCl density gradient centrifugation. This preparation was then sequenced to verify the correct insertion and correct sequence of the IL-18 fusion construct. Control plasmids, expressing either OVA alone, or an irrelevant sequence (single-chain Fv), have also been described previously (Maecker et aL, supra.) Finally, a plasmid expressing IL-18 alone was produced for this study. The IL-18 sequence was moved into another vector, pTCAE 5.3, by PCR amplification that added restriction sites Dralll and Hpal. From this vector, the insert was excised with Dralll and Hpal digestion. An OVA-IL-6 expressing plasmid, pOVA-IL-6, was digested with Dralll and BamHI to remove the entire OVA-IL-6 insert. The IL-18 insert described above was then ligated into this vector backbone via Dralll; the remaining sticky ends were blunted by use of T4 polymerase, and joined by blunt-end ligation. The IL-18 insert was checked by DNA sequencing as above.

IL-18 Bioactivity Assay. To determine IL-18 bioactivity of the OVA-IL-18 fusion construct, the recombinant protein was tested for induction of IFN_{γ} production by a murine Th1 cell line, DOH2. First, plasmids expressing OVA-IL-18 or, as a control, OVA-IL-4, were transfected into COS-7 cells using DEAE-dextran in a standard method. Supernatants from the transfected cells were harvested after 3 days and stored at 4° C. The murine Th1 cell line, DOH2, was produced and maintained as described previously. DOH2 cells were resuspended at 5×10⁵ per ml in DMEM medium with 10% fetal bovine serum. 100 μl of cell suspension were plated per well of a microtiter plate, along with 100 μl of media or COS-7 supernatant at dilutions of 1:2, 1:4, 1:8, or 1:16. The cells were incubated at 37° C. for 48 h, then supernatants harvested from each well and tested for IFN_{γ} production by ELISA. Shown in FIG. 1B, only OVA-IL-18 containing supernatant generated IFN_{γ} production from DOH2 cells, in a dose-dependent manner.

Immunization Protocols.

Prevention of AHR (FIG. 1C, top): On day zero, BALB/c mice were injected intramuscularly (i.m.) in the quadriceps muscles with 100 μg of each plasmid DNA in a final volume of 100 μl 0.9% NaCl, divided bilaterally. On day 17, the mice were boosted i.m. with the same amount of plasmid DNA. The mice were then sensitized to ovalbumin (OVA) protein using an established protocol for the induction of airway hyperreactivity in BALB/c mice (Hansen et al. (1999) J. Clin. Invest. 103:175). OVA (50 μg) adsorbed to 2 mg aluminum potassium sulfate (alum) was administered intraperitoneally (i.p.) on days 24 and 38, followed by 50 μg OVA in 50 μl PBS given intranasally (i.n.) on days 38, 49, 50 and 51. Control mice received i.p. injections of alum alone and intranasal PBS. One day after the last intranasal challenge (day 52), AHR was measured in conscious mice after inhalation of increasing concentrations of methacholine (see below). Within 5 days of the last challenge, blood was taken, mice were sacrificed, lungs were removed and fixed, and splenocytes were isolated for in vitro culture.

ii. Antigen specificity test: In experiments to determine whether the effects of the different DNA constructs on the immune response of BALB/c mice were antigen-specific, mice were first injected with the different OVA DNA constructs i.m. (see above). One week later, the mice were immunized in the footpads either with the relevant antigen, OVA (200 μg/mouse), or an irrelevant antigen, Keyhole Limpet Hemocyanin (KLH, 100 μg/mouse), each emulsified in IFA. After seven days the mice were sacrificed, and lymphocytes were isolated from the draining lymph nodes (LN) for in vitro culture.

iii. Treatment of mice with anti-cytokine and depletion mAb: BALB/c mice were injected i.p. with 1 mg of mAb XMG1.2 (for IFN_{γ} depletion), 200 μg of mAb 53.6.7 (for CD8 depletion) or 1 mg of mAb LC4 (control mAb) every other day for six days, then every fifth day thereafter, starting five days before immunization with DNA. Antibody injection was continued until the immunization protocol was finished. Blood was collected on the day of sacrifice and stained with anti-mouse CD8-PE and anti-mouse CD4-FITC mAb (Pharmingen Corp., San Diego, Calif.). FACS analysis revealed a ˜90% depletion of CD8⁺ cells in anti-CD8 mAb treated mice in each of two replicate experiments.

iv. Reversal of established AHR (FIG. 1C, bottom): To investigate whether DNA immunization can reverse established AHR rather than inhibit the development of AHR, BALB/c mice were first sensitized with OVA prior to vaccination with the DNA plasmids. 50 μg OVA adsorbed to alum was administered i.p. once on day zero. 50 μg OVA in 50 μl PBS was administered i.n. on days 8 and 9. On days 10 and 25 the different DNA constructs were injected i.m. in the quadriceps muscles (100 μg in 100 μl 0.9 % NaCl). On day 39 the mice were boosted again with OVA i.n., and AHR was measured one day later (day 40). Mice were sacrificed and bronchial LN cells isolated for in vitro culture within 5 days of the last OVA challenge.

Measurement of Airway Responsiveness. Airway responsiveness was assessed by methacholine-induced airflow obstruction from conscious mice placed in a whole body plethysmograph (model PLY 3211, Buxco Electronics Inc., Troy, N.Y.). Pulmonary airflow obstruction was measured by Penh using the following formula:

Pₑₙₕ=((Te/RT−1)×(PEF/PIF),

where Pₑₙₕ=enhanced pause (dimensionless), Te=expiratory time, RT=relaxation time, PEF=peak expiratory flow (ml/s), and PIF=peak inspiratory flow (ml/s). Enhanced pause (Penh), minute volume, tidal volume, and breathing frequency were obtained from chamber pressure, measured with a transducer (model TRD5100) connected to preamplifier modules (model MAX2270) and analyzed by system XA software (model SFT 1810). Measurements of methacholine responsiveness were obtained by exposing mice for 2 min to aerosolized 0.9% NaCl, produced by a sonicator (Portable Ultrasonic, 5500D, DeVilbiss Health Care, Inc. Sommerset, Pa.), followed by incremental doses (2.5-20 mg/ml) of aerosolized methacholine. Results were expressed for each methacholine concentration as the percentage of baseline Penh values after 0.9% NaCl exposure.

OVA-Specific IgE Assay. Mice were bled at the time of sacrifice and OVA-specific IgE was determined using a modified Ag-specific ELISA. Plates were coated overnight with rat anti-mouse IgE mAb EM95 (5.0 μg/ml). After washing and blocking, samples were applied and incubated overnight. Plates were again washed and biotinylated OVA (10 μg/ml) was added. Two hours later, plates were washed and HRP-conjugated streptavidin (Southern Biotechnology Associates, Birmingham, Ala.) was added. Plates were developed with OPD substrate and the OD determined at 492 nm. Serum from mice hyperimmunized with OVA in alum was standardized for IgE levels against an anti-OVA IgE mAb. This serum was used as a standard in the OVA-specific IgE ELISA.

Restimulation of Spleen and LN Cells in vitro. Spleens or bronchial LN were removed, depleted of resting B cells by adherence to goat anti-mouse Ig-coated plates, and 4×10⁵ cells were restimulated in vitro with OVA (100 μg/ml) or KLH (10 μg/ml). Cells were cultured in 96 well microtiter plates in 150 μl medium. Supernatants were harvested after four days for determination of IL-4 and IFN_{γ} levels. Cytokine content in each sample was measured in triplicate by ELISA.

Cytokine ELISA. ELISA were performed as previously described (Macaulay et al. (1997) J Immunol 158:4171). The mAb pairs used were as follows, listed by capture/biotinylated detection mAb: IFN_{γ}, R4-6A2/XMG1.2; IL-4, 11B11/BVD6-24G2. A standard curve using recombinant cytokine in 1:2 dilutions from 20-0.156 ng/ml for IFN_{γ}, or 500 to 7.5 pg/ml for IL-4, was used for quantitation.

Collection of bronchio-alveolar lavage (BAL) fluid. Animals were sacrificed by CO₂ asphyxiation. The trachea was cannulated, and the lungs were lavaged four times with 300 μl of 1% BSA in 1X PBS. Cells in the lavage fluid were then counted using a hemocytometer, and BAL cell differentials were determined on slides preparations stained with Hansel Stain (Lide Laboratories, Florissant, Mo.). At least 200 cells were differentiated by light microscopy based on conventional morphogenic criteria.

Results

IL-18 bioactivity of fusion construct. To test the bioactivity of IL-18 in the OVA-IL-18 fusion protein, COS-7 cells were transiently transfected with plasmid DNA and the cells cultured for 4 d. The supernatants of these cell cultures were then tested for IL-18 bioactivity, via the ability to induce IFN_{γ} production from a Th1 cell line. FIG. 1B shows that supernatant from OVA-IL-18 transfected COS-7 cells induced IFN_{γ} production from the established Th1 cell line, DOH2, while medium or supernatant from control OVA-IL-4 transfected cells did not, indicating that protein produced from the IL-18 plasmid had biological activity.

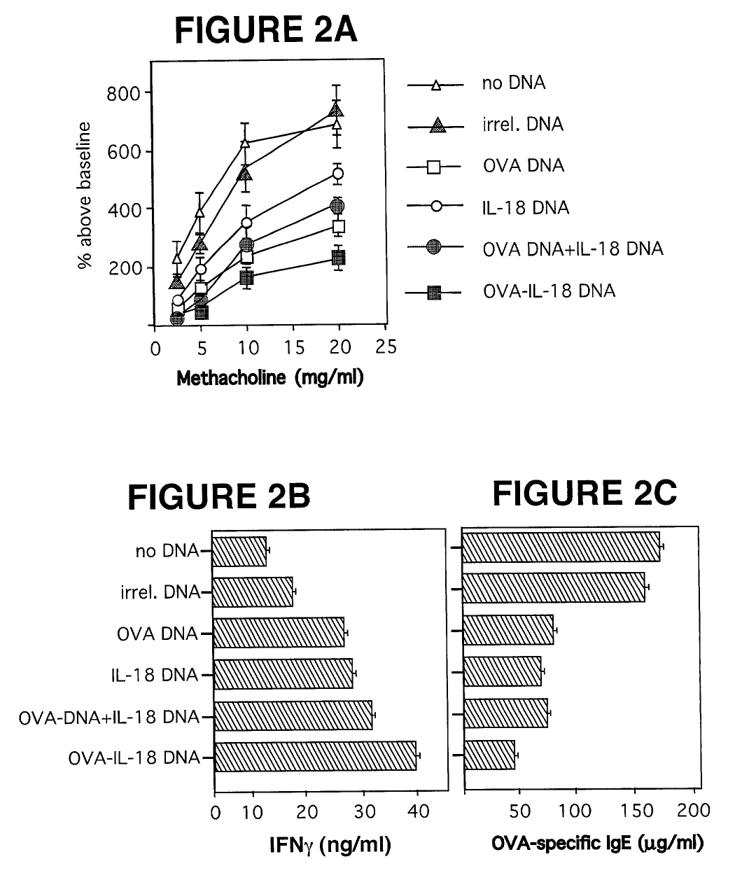

Inhibition of AHR by vaccination with different DNA vectors. Having established bioactivity of the OVA-IL-18 fusion construct, it was next tested in vivo for its ability to inhibit AHR in a murine asthma model. BALB/c mice were vaccinated i.m. with irrelevant DNA, OVA DNA, IL-18 DNA, a mixture of OVA DNA and IL-18 DNA, or the OVA-IL-18 DNA fusion construct. The mice were then sensitized for AHR with i.p. and i.n. administrations of OVA. One day after the last OVA challenge, AHR was measured in response to increasing concentrations of methacholine in conscious mice placed in a whole body plethysmograph. FIG. 2A demonstrates that sensitization of mice with OVA resulted in the development of significant AHR when the mice were challenged with methacholine. Mean Penh values were calculated and data expressed as a percent above baseline (NaCl-induced AHR). Error bars represent S.E.M. of 5 animals per group. Vaccination of mice with the OVA-IL-18 DNA fusion construct dramatically inhibited the development of AHR. Vaccination with OVA DNA, OVA DNA+IL18 DNA, or IL-18 DNA also inhibited development of AHR, although to a lesser extent. Injection of irrelevant DNA had no effect on the OVA-induced AHR.

Effects of DNA vaccination on cytokine production. A known property of both IL-18 and DNA vaccination in general is the ability to induce IFN_{γ} production in vivo. To determine whether the reduced AHR in mice vaccinated with the OVA-IL18 plasmid correlated with alteration of cytokine profiles in CD4+ T cells, mice were sacrificed after measurement of airway reactivity. Spleen cells were removed and stimulated with OVA in vitro. FIG. 2B shows that DNA vaccination significantly increased OVA-specific IFN_{γ} production in OVA-immunized mice. Mice were sacrificed within 5 days of the last challenge, and spleen cells were removed and cultured with 100 μg/ml OVA. IFN_{γ} levels in the supernatants were determined by ELISA. The strongest IFN_{γ} increase was induced by the OVA-IL-18 DNA fusion construct. The increase of IFN_{γ} production was comparable in the groups that received either OVA DNA or IL-18 DNA alone, and was slightly higher after injection of the mixture of OVA DNA and IL-18 DNA. While vaccination with OVA DNA, IL-18 DNA, or a mixture of OVA DNA and IL-18 DNA also induced IL-4 production, the OVA-IL-18 DNA fusion construct did not increase IL-4 levels in OVA-immunized mice. Irrelevant DNA had no significant effect on IFN_{γ} or IL-4 production.

Inhibition of IgE synthesis by DNA vaccination. We also analyzed the levels of anti-OVA IgE Ab responses in serum collected from these mice, OVA-specific IgE was very high in OVA-immunized BALB/c mice (FIG. 2C). Vaccination with the different DNA vectors before immunization with OVA significantly reduced the level of OVA-specific IgE. The inhibitory effect on IgE production was strongest with the OVA-IL-18 fusion construct and did not differ significantly between OVA DNA, IL-18 DNA, or the mixture of OVA-DNA and IL-18 DNA. In contrast, irrelevant DNA had little effect on IgE production.

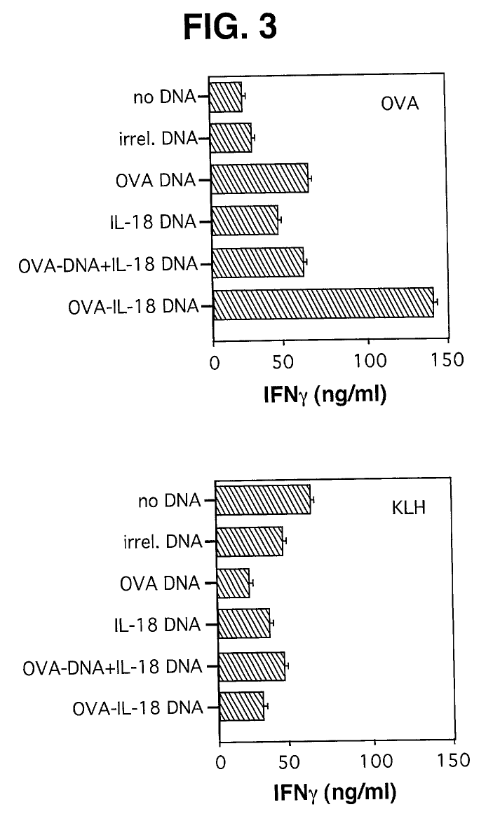

Specificity of DNA vaccination. To test whether the effects of immunization with OVA-IL-18 DNA were antigen-specific, we boosted DNA vaccinated mice after one week with either the relevant protein (OVA) or with an irrelevant protein, Keyhole Limpet Hemocyanin (KLH). After 7 days, spleens were removed and splenocytes cultured with the antigen used for boosting. FIG. 3, left panel shows that in OVA protein boosted mice, the increase in IFN_{γ} was most notable in the group that received the OVA-IL-18 DNA fusion construct. In contrast, in mice boosted with the irrelevant antigen KLH, IFN_{γ} production was not increased by vaccination with OVA-IL-18 DNA, and was similar in all groups receiving the various OVA DNA constructs (FIG. 3, right panel). These results indicated that vaccination with the OVA-IL-18 DNA construct greatly enhanced IFN-_{γ} production, but the effect on IFN-_{γ} production was confined to the OVA-specific response.

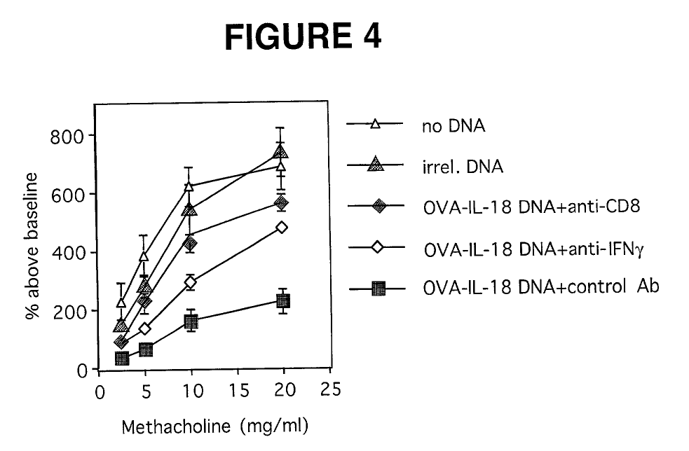

Inhibition of AHR depends upon CD8⁺ T cells and IFN_{γ}. To investigate the mechanism by which vaccination with OVA-IL18 affected OVA-specific responses, we administered blocking antibody to IFN_{γ} or depleting antibody to CD8+ T cells during the immunization protocol. As expected, mice that received irrelevant DNA and control mAb developed strong AHR, which was significantly reduced in mice vaccinated with OVA-IL-18 DNA in the presence of control mAb (FIG. 4). Treatment with anti-CD8 mAb largely restored AHR in the OVA-IL-18 DNA immunized animals. Anti-IFN_{γ} mAb also restored AHR, although to a lesser extent than that seen with CD8 depletion. Thus, inhibition of AHR by OVA-IL-18 DNA was dependent upon both IFN_{γ} production and the presence of CD8⁺ cells.

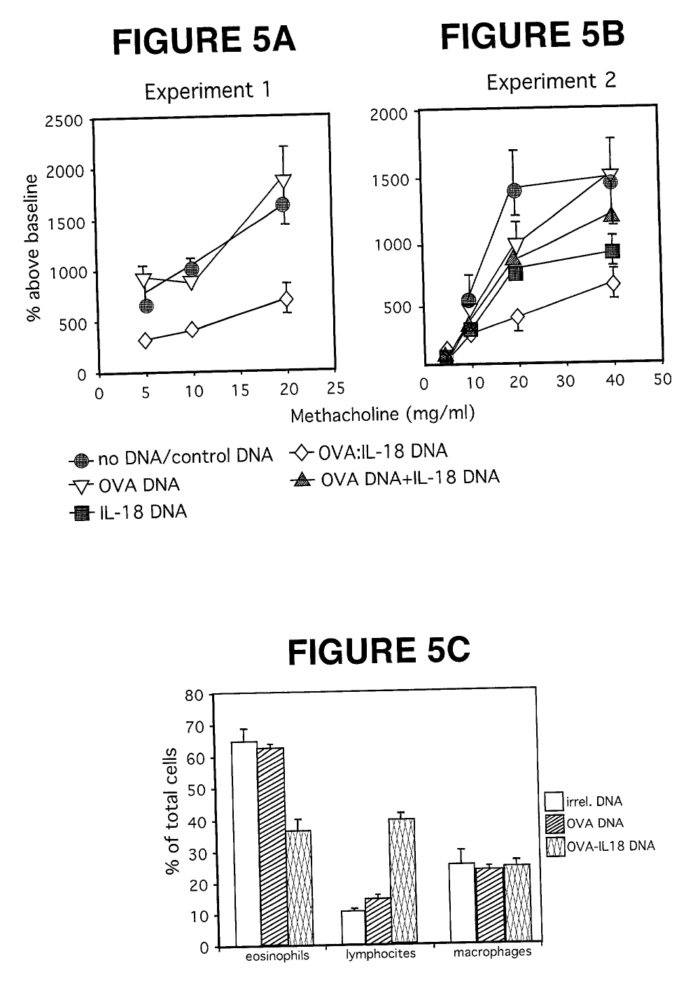

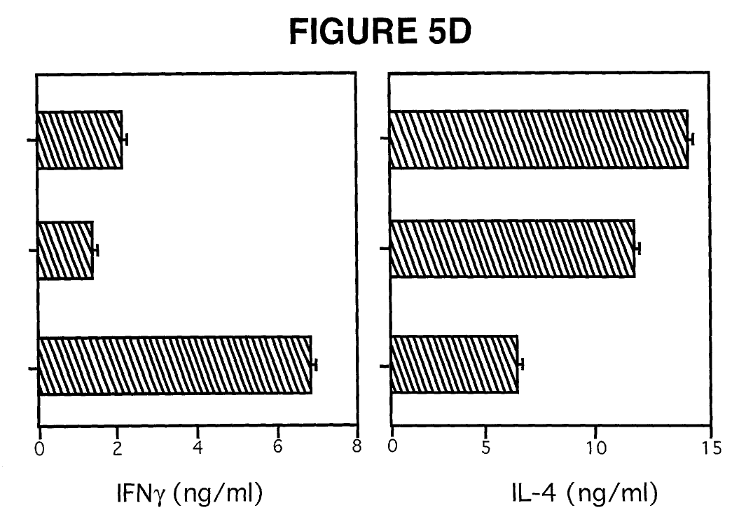

Reversal of AHR by DNA vaccination. To determine whether DNA vaccination could reverse established AHR in addition to inhibiting the development of AHR, mice were first sensitized with OVA protein, by administering OVA i.p. in alum, and OVA i.n. to establish AHR in these mice. The mice were then vaccinated either with OVA-IL-18 DNA, OVA DNA,IL18 DNA, OVA DNA 1 IL18 DNA, or irrelevant DNA (as indicated in FIG. 5A), and AHR was measured after a final i.n. OVA boost. Mice that received irrelevant DNA developed strong AHR (FIG. 5A and 5B). In contrast, vaccination of the mice with the OVA-IL-18 DNA construct greatly reduced AHR. Under these conditions of pre-established AHR, the OVA-IL-18 DNA alone was significantly more effective than OVA DNA+IL18 DNA, IL18 DNA alone, or OVA DNA alone.

The reduction of AHR was consistent with the examination of BAL fluid, in which OVA-IL-18 DNA, but not OVA DNA greatly reduced the percent of eosinophils in BAL fluid (FIG. 5C). Eosinophils were still present in the lungs of mice treated with OVA-IL-18 DNA, perhaps consistent with the fact that some degree of AHR was present in these mice, and with the observation that IL-18 can recruit eosinophils into the airways. These data demonstrate that: (1) OVA-IL-18 cDNA but not OVA cDNA reverses ongoing AHR in previously sensitized mice; and (2) the activity of OVA-IL-18 DNA is clearly superior to that of OVA DNA in such sensitized animals.

IL-4 and IFN_{γ} measurements in OVA-immunized mice before and after DNA vaccination. Mice vaccinated with DNA constructs after establishment of OVA-induced AHR were sacrificed, and cytokine profiles of bronchial LN cells were analyzed. Vaccination with OVA-IL-18 DNA resulted in a dramatic increase of IFN_{γ} production in bronchial LN cells as compared to animals receiving irrelevant DNA or OVA DNA alone (FIG. 5C). Vaccination with OVA-IL-18 DNA also reduced OVA-specific IL-4 production compared to the other DNA constructs (FIG. 5D). In addition, the OVA-IL-18 plasmid was much more effective than the OVA plasmid in reducing OVA specific IgE production (OVA plasmid treated group, 5690±800; OVA-IL-18 plasmid treated group, 2968±81 ng/ml). These experiments demonstrated that OVA-IL-18 DNA, but not OVA DNA could boost OVA-specific IFN_{γ} production and reduce IL-4 and IgE production, even when given in the context of ongoing AHR.

IL-4 and IFN_{γ} measurements in OVA-immunized mice before and after DNA vaccination. Mice vaccinated with DNA constructs after establishment of OVA-induced AHR were sacrificed, and cytokine profiles of bronchial LN cells were analyzed. Vaccination with OVA-IL-18 DNA resulted in a dramatic increase of IFN_{γ} production as compared to animals receiving irrelevant DNA or OVA DNA alone (FIG. 5C). Vaccination with OVA-IL-18 DNA also reduced OVA-specific IL-4 production compared to the other DNA constructs. These experiments demonstrated that OVA-IL-18 DNA, but not OVA DNA could boost OVA-specific IFN_{γ} production even when given in the context of ongoing AHR.

The results presented above demonstrate that an OVA-IL-18 fusion DNA construct was highly effective in preventing and reversing allergen-induced AHR in a murine asthma model. Previous studies have demonstrated the usefulness of allergen DNA immunization in the prevention of allergic diseases and AHR, but allergen DNA vaccination has not been previously reported to successfully reverse ongoing AHR. We now describe an OVA-IL-18 DNA construct that effectively corrected established AHR in an allergen-specific fashion when administered only twice. While both the OVA-IL-18 and the OVA DNA constructs, when administered to naive mice, prevented the subsequent induction of AHR and reduced allergen-specific IgE production, the OVA-IL-18 DNA was unique in its capacity to reverse established AHR. The protective effects of OVA-IL-18 appeared to be mediated by IFN_{γ} and CD8 cells, presumably induced by IL-18 and by the CpG motifs present in the vector backbone. These results demonstrate that the addition of IL-18 to allergen DNA constructs substantially improves the efficacy of allergen DNA immunization, and suggest that vaccination with allergen-IL-18 DNA may be clinically effective in the treatment of patients with ongoing chronic allergic asthma.

The potent inhibitory effects of OVA-IL-18 DNA vaccination on AHR and IgE production was dependent on the fusion of the cytokine and allergen. Thus, codelivery of non-fused OVA DNA and IL-18 DNA was much less effective compared to the fusion construct vector in inducing IFN_{γ} production, reducing IgE production, and preventing the development of AHR. This indicated that the fusion of the cytokine and the antigen was crucial for protection in this model. Vaccination with OVA-IL-18 DNA maximized the salutary IL-18 effects for asthma, presumably by focusing the activity of IL-18 onto OVA-specific T cells and B cells. The strategy of delivering IL-18 conjugated with antigen is particularly applicable to allergic disease, since the major allergens (and in many instances the major allergenic proteins) have been identified.

The inhibitory effect of OVA-IL-18 DNA on AHR was dependent on the presence of CD8⁺ T cells, since the protective effects of OVA-IL-18 DNA could be almost completely reversed by depletion of CD8⁺ T cells. This observation supports other studies demonstrating the important role of CD8⁺ T cells in asthma. For example, Hsu et al. demonstrated that the protective effect of allergen DNA vaccination could be transferred with CD8⁺ T cells. Furthermore, animal experiments have revealed that CD8⁺ T cells regulate IgE production and allergen-induced AHR (McMenamin et al. (1993) J. Exp. Med. 178:889; Renz et aL. (1994) J Immunol 152:351). The induction of regulatory CD8 cells may have been enhanced by the potent capacity of IL-18 to induce CD8 T cells, and by the administration of OVA as cDNA, which may skew antigen presentation through an endogenous pathway. It is well established that peptides derived from intracellular antigens are generally presented to CD8⁺ T cells by major histocompatibility complex (MHC) class I molecules, and this antigen presenting pathway may be important in the induction of regulatory CD8 cells, when allergen cDNA is administered intramuscularly.

The inhibitory effect of OVA-IL-18 DNA on AHR was also partially dependent on IFN_{γ} activity, since coadministration of anti-IFN_{γ} mAb partially prevented the effects of OVA-IL-18 DNA. Both IL-18 as well as CpG motifs present on the vector backbone effectively induce IFN_{γ} production, which has been shown in studies with direct mucosal IFN_{γ} gene transfer to inhibit both the induction of antigen- and Th2-cell-induced pulmonary eosinophilia and AHR. In addition, CpG motifs induce IL-12 production, important not only in enhancing the induction of IFN_{γ}, but also in promoting the expression of IL-18 receptors on T cells, and in inhibiting antigen-induced airway eosinophilia and bronchial hyperreactivity in a murine model.

Only two injections of OVA-IL-18 DNA were sufficient to reverse established AHR, suggesting that such an approach is clinically useful for the treatment of chronic allergic disease and asthma. Currently, conventional allergen immunotherapy, performed by the subcutaneous injection of increasing doses of allergen, is used to treat patients with allergic disease. However, such therapy is inefficient, requiring nearly 100 injections over a period of 3-5 years, and it is associated with frequent allergic reactions, including anaphylaxis. Nevertheless, conventional allergen immunotherapy is the only currently available therapy that, when successful, alters the underlying pathologic allergen-specific Th2 driven responses, resulting in clinical tolerance to subsequent allergen exposure. DNA vaccination may be a safer form of allergen immunotherapy, particularly since DNA-based immunization provides prolonged, endogenous expression of antigen. Plasmids have been found to persist episomally in muscle cells, and gene expression in the skeletal muscle and persistent immunity to the antigen can be detected for more than a year after injection. Moreover, allergen-IL-18 DNA constructs may be considerably more potent in down-modulating Th2 biased immune responses than conventional allergen extracts, and may thus provide rapid, effective and potentially curative therapy for allergic disease and asthma.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of enhancing a Th1 T cell mediated immune response, the method comprising:
administering an effective dose of a fusion protein of IL-18 and an antigen associated with said response;
wherein the response of said Th1 T cell is enhanced.

2. A method of diminishing a Th2 T cell mediated antigen-specific allergic response, the method comprising:
administering an effective dose of a DNA construct encoding a fusion protein of IL-18 and an antigen associated with said allergic response for a period of time sufficient to diminish said antigen-specific allergic response.

3. The method according to claim 2 wherein said antigen-specific allergic response is IgE-mediated allergic asthma.

4. The method according to claim 2 wherein said antigen-specific allergic response is IgE-dependent allergic rhinoconjunctivitis.

5. The method according to claim 2 wherein said antigen-specific allergic response is IgE-mediated anaphylactic reactions.

6. The method according to claim 2 wherein said diminished response is mediated by CD8⁺ T cells.

7. The method according to claim 2, wherein said diminished response is characterized by decreased antigen-specific synthesis of IgE, and increased synthesis of _{γ}-interferon.

8. A method of treating asthma associated allergies, the method comprising:
administering an effective dose of a DNA construct encoding a fusion protein of IL-18 and an antigen associated with said asthma for a period of time sufficient to diminish said asthma associated allergies.

9. The method according to claim 8, wherein said asthma associated allergies are ongoing at the time of said administering step.

10. A pharmaceutical composition for the treatment of allergies, comprising:
a DNA construct encoding a fusion protein comprising an allergy associated antigen and interleukin 18; and
a pharmaceutically acceptable carrier.

11. The pharmaceutical composition of claim 10, wherein said DNA construct further comprises immunostimulatory DNA sequences.
